(19) United States
(12) Patent Application Publication
(10) Pub. No.: US 2016/0166694 A1
(43) Pub. Date: Jun. 16, 2016

(54) PHARMACEUTICAL COMPOSITION, METHOD OF PREPARATION AND METHODS OF TREATING ACHES/PAINS

(71) Applicant: AVANTI PHARMA CORPORATION, Boca Raton, FL (US)

(72) Inventors: Nicholas DeSica, Parkland, FL (US); Muhammed Ali, Fairfield, CA (US); Richard Li, Fairfield, CA (US)

(21) Appl. No.: 15/054,081

(22) Filed: Feb. 25, 2016

Related U.S. Application Data
(63) Continuation-in-part of application No. 13/898,130, filed on May 20, 2013, now abandoned.
Application No. 13/898,130 is a continuation-in-part of application No. 12/437,370, filed on May 7, 2009, now Pat. No. 8,445,545.
(60) Provisional application No. 61/051,090, filed on May 7, 2008.

(51) Int. Cl. A61K 47/10; A61K 9/00; A61K 9/14; A61K 36/9068; A61K 47/26; A61K 47/18; A61K 36/28; A61K 31/192; A61K 47/02

(52) U.S. Cl. CPC A61K 47/10; A61K 31/192; A61K 9/006; A61K 9/145; A61K 47/02; A61K 47/26; A61K 47/183; A61K 36/28; A61K 36/9068

(57) ABSTRACT

Provided are methods and compositions useful for treating/aches and/or pains. The compositions include ibuprofen in a clear stable aqueous system of water, alcohol and glycerin that is stable at room temperature for at least 6 months without separation or precipitation. The composition is effective for delivering ibuprofen directly through the mucosal or buccal tissue without passing through the GI tract.

### CROSS-REFERENCE TO RELATED APPLICATION

This application is a continuation-in-part of Ser. No. 13/898,130 filed May 20, 2013, which is a continuation-in-part of Ser. No. 12/437,370 filed May 7, 2009, now U.S. Pat. No. 8,445,545, issued May 21, 2013, which claims the benefit of Provisional Application No. 61/051,090, filed May 7, 2008, which are hereby incorporated by reference in their entirety.

### FIELD OF THE INVENTION

The present invention relates to a stable aqueous pharmaceutical analgesic composition comprising ibuprofen having an average particle size of about 0.01 to 500 nm. The invention is also directed to a method of producing a stable aqueous-ethanol ibuprofen composition. The composition can be a pharmaceutical composition for administering to a patient for the treatment of aches, pains and/or inflammation in muscles, joints and/or tissues.

### BACKGROUND OF THE INVENTION

Aches, pains, and discomfort are common problems. For example, ibuprofen has the chemical name 2-(4-Isobutylphenyl)propionic acid and is a well-tolerated drug possessing analgesic, antipyretic and anti-inflammatory activities (Merck Index, 11th edition, no. 4812). Current treatments for such pains are pills, gelatin capsules, and powder capsules that make their way through the gastrointestinal tract to the circulatory system. Such a traversal of the various organs in the body depletes the active ingredients in the liver, stomach and intestines while exposing the tissues of those organs to the effects of the active ingredient. Some patients have experienced stomach irritation and ulcers from orally ingested treatments. Some adults and many young children also have difficulty swallowing such pills and actively seek other forms of such medication. Other ways to administer analgesic medications and treatments include subcutaneous injections and nasal sprays. Subcutaneous injections are painful and difficult to self-administer. Nasal sprays have hitherto experienced stability problems with the dispersion and integrity of the active ingredients.

A number of analgesic and therapeutic medications and homeopathic treatments are known and reflected in one or more patents. The interest in homeopathic and/or herbal medicines has increased recently due in part to the lower cytotoxicity associated with such medications. Homeopathy is commonly used to mean a system of medicine based on the use of infinitesimal doses of medicines capable of producing symptoms similar to those of the disease treated. By stimulating a subject's natural defenses (i.e., increasing the symptoms) the subject will be motivated or directed towards homeostasis, since one's symptoms are actually efforts of the organism to reestablish homeostasis or balance. Homeopathic treatment encompasses some forms of natural materials including plant extracts and the like. However, some natural plant extracts are not necessarily homeopathic treatments as the extracts themselves do not stimulate disease or disorder symptoms but rather inhibit their onset or severity.

For example, ginger has been used with some success for relief of nausea. The administration of 1,000 to 2,000 mg of ginger orally by tablet has been found to effectively reduce nausea in the case of motion sickness.

Another example is feverfew, an herb that is widely available and has been investigated in modem times. Historically, feverfew is known to have been used in the treatment of fevers, from whence it derives its name, and also in rheumatic conditions. Fresh feverfew leaves have sometimes been chewed by subjects wishing to rid themselves of migraine. However, a common adverse effect reported by those who have used this technique is the generation sores in the mouth and sensitization of oral tissues. Additionally, many patients find this mode of administration to be crude and unpleasant. Feverfew tablets or capsules do not expose the mouth tissues to the same effects as chewed leaves and have been employed by practitioners of herbal medicine.

The use of feverfew for treatment of migraines is known. For example, U.S. Pat. No. 6,103,218 to Brucker, et al. discloses a composition and delivery system in which feverfew is delivered in the form of an aqueous nasal spray for the relief of migraine headaches.

The use of a combination of feverfew and ginger for the treatment migraines in a sublingual form is known. U.S. Patent Application Publication No. 2006/0222722 to Roberts, et al. discloses sublingual methods of treating arthritis by administering a composition including feverfew and ginger. The reference lacks disclosure of combining ibuprofen in the composition and moreover, the ability to make a stable, sublingual pharmaceutical spray composition in which an ingredient of the composition is a stable, water-soluble ibuprofen.

The combination of analgesics and feverfew dissolved in aqueous mediums for treating aches and/or pains has also been suggested. U.S. Pat. No. 6,770,263 to Brucker discloses a composition that comprises an aqueous medium in which feverfew and an analgesic are dissolved or dispersed. One problem associated with such a composition is that the analgesic, such as ibuprofen, is not stable for extended periods and through a wide variety of temperature extremes.

Liquid formulations for delivering medicaments and herbal therapeutic agents is not, however, a new development. Biologically active agents such as nutritional supplements, hormones, and a variety of pharmaceutical preparations are typically provided in oral (liquids or solids) or injectable dosage formulations. There are, however, difficulties with maintaining stability of the solution or dispersion without precipitation and with maintaining efficacy of the ingredients associated with this formulation.

One manner to overcome the limitations discussed above is to produce granulates from powder mixtures. For purposes of administration, these granulates are usually converted into tablets, enclosed in capsules or in sachets. It has also been long known that granules or tablets can be coated with films, which can serve to delay the release of the active ingredient they contain, disguise an unpleasant taste, and/or improve the stability of the composition. A major limitation of the use of such coated granulates in liquid formulations is that it has been difficult to obtain particles of an appropriate size to enable them to be easily suspended and kept in suspension in the fluid vehicle.

One manner of producing granules involves the use of a conventional mixer-granulator, which consists of a vessel, which may be of varying shape, equipped with an agitator that keeps the powder moving while the granulation fluid is being added. The motion is slow and the resulting globules, even though suitable for making conventional dosage forms such as tablets or capsules, does not possess the density, shape and particle-size distribution suitable for subsequent coating.

Unlike conventional mixer-granulators, extruder-spheronizers can produce spherical particles of homogeneous sizes and even shapes and surfaces. The limitation that prevents their application to micro granulates suitable for liquid suspensions is the average product size, which is rarely smaller than 1-2 mm and in any case never smaller than 500 μm.

It would be desirable to have a method for producing analgesic and similar pain-treating compositions that could produce particles of very fine size in a stable, aqueous formulation.

It would also be desirable to have a spray formulation that would require low concentrations of active ingredients for contact with sublingual mucosal tissues.

### SUMMARY OF THE INVENTION

It is an objective of the invention to provide a composition as well as a process for making the composition, having a super-fine particle size that can form a stable aqueous ibuprofen composition in the form of a dispersion and/or solution.

The aqueous ibuprofen composition of the invention is a stable composition containing an amount of ibuprofen for treating a patient in need thereof. The composition of the invention may contain an aqueous-ethanol and glycerin solvent and carrier, an emulsifier and a buffering agent. The solvent system of the ibuprofen composition is an aqueous based liquid including an amount of an organic solvent such as glycerin. The emulsifier can be lecithin, polysorbate, POLYSORBATE 20 and mixtures thereof. The solvent/carrier of the composition is preferably a mixture of water, ethanol and glycerin where the ethanol is present in an amount of not more than 20 wt % and more preferably of not more than 15 wt %.

In one embodiment of the invention the ibuprofen composition is a composition suitable for oral delivery through the mucosal tissue such as by an oral liquid composition or a sublingual or buccal composition. The sublingual or bucal composition preferably includes a mucosal permeation enhancing agent. The mucosal permeation enhancing agent can be polysorbate, a polyoxyethylene such as POLYSORBATE 20 and potassium sorbate. A buffering agent such as arginine can also be included in the composition.

The aqueous ibuprofen composition of the invention is prepared by mixing the various components under conditions to obtain the aqueous composition where the ibuprofen is stable in the composition without separation, precipitation or settling for at least 6 months. In one embodiment the aqueous system includes glycerin as co-solvent. The aqueous ibuprofen composition is prepared by forming a mixture of water and glycerin in a first vessel. A mixture of the emulsifier and the mucosal permeation enhancing agent can be prepared in a second vessel. A solution of ibuprofen is prepared by dissolving ibuprofen in an alcohol. The alcohol is preferably ethanol. The ethanol can be an aqueous mixture of water with ethanol where the ethanol is present in an amount sufficient to dissolve the ibuprofen. In one embodiment of the invention the ethanol is about 95% ethanol. The ibuprofen/ethanol solution is combined with the water and glycerine mixture and continuously mixed in a high shear mixer under a vacuum to form a uniform mixture. The mixture is mixed for about 30 minutes under high shear for sufficient time to form the aqueous ibuprofen composition where the ibuprofen is dispersed in the aqueous system. The ibuprofen preferably has a particle size of less than 500 microns and preferably less than about 100 microns.

In accordance with this and other objectives of the invention that will become apparent from the description herein, the present invention provides a composition having dispersed or dissolved therein (a) an analgesic preparation including ibuprofen and (b) an optional herbal therapeutic agent comprising a mixture of feverfew and ginger, wherein the average particle size of the analgesic preparation and the herbal therapeutic agents is about 0.01 to 500 nm, and preferably about 0.01 to 100 nm.

The invention also provides a method of treating a subject disposed to aches, pains and/or tissue inflammation including headache, migraine, toothache, earache, joint pain, backache, abdominal cramps, and the like by administering an effective amount of the aqueous ibuprofen composition wherein relief from the pain and/or tissue inflammation occurs within minutes of the administering.

As described more fully herein, the invention also provides a method of forming a stable, water-soluble composition suitable for sublingual, buccal, or via the gastrointestinal tract—administration that includes the steps of: (a) dissolving ibuprofen USP grade powder, preferably with a dissolution solution that includes water alcohol, and glycerin to form a dissolved ibuprofen solution; (b) transferring said dissolved ibuprofen solution to a vacuum equipped vessel having a high shear mixer mounted thereon; and (c) mixing said dissolved ibuprofen solution under high shear mixer for a time sufficient to reduce the average particle size of said dissolved ibuprofen to less than or equal to 500 nanometers. Preferably, the process also contemplates (d) allowing the mixture from step c) to rest for 10-20 minutes and (e) equilibrating the product for approximately 24 hours.

Also contemplated herein is a method of treating a subject disposed to a disorder having symptoms that include aches, pains and/or tissue inflammation is described that includes: administering to a subject via sublingual, buccal, or the gastrointestinal tract, an effective amount of an aqueous ethanol, glycerin composition containing a effective amount of ibuprofen having an average particle size of about 500 nanometers or less, and preferably 100 nm or less.

The invention further provides a method of producing a sublingual composition comprising therein an analgesic such as, for example, ibuprofen, wherein the sublingual composition is tolerant to mucus membranes and can be used as a spray.

In one aspect of the present invention, a composition according to the invention comprises ibuprofen having an average particle size of less than about 500 nm in an aqueous formulation such as a spray formulation. In this embodiment, the composition comprises ibuprofen as well as other ingredients in the formulation to provide a composition suitable for administration of the composition of ibuprofen sublingually. The optional ingredients can include ginger extract, feverfew, flavorings, colorants and the like.

The present invention provides a commercially viable method for making a highly effective, stable analgesic composition that can be administered via sublingual, buccal, or the gastrointestinal tract. The composition is stable through a variety of conditions and permits administration of effective pain and/or inflammation treatments to a wide variety of patients under even the most adverse and unsanitary circumstances. The composition contains ibuprofen in solution and nanometer size particles in a stable suspension so that the ibuprofen can be absorbed directly through the mucous membranes and buccal tissue for rapid delivery to the bloodstream without the need to pass through the GI track, without undergoing portal circulation or undergo peptic metabolism.

The mucosal tissue does not contain the acids and enzymes present in the gastrointestinal tract. Thus, via the sublingual route, the herbal medicines disclosed herein, as well as the analgesic, are readily available for absorption into the blood stream and related tissues of the subject without unwanted degradation. Accordingly, smaller amounts of such herbal medicines and/or analgesics are useful in order to treat a subject afflicted with a disease or disorder causing aches, pains, and/or discomfort. The smaller amounts used in the compositions and methods of the invention also result in less gastrointestinal discomfort, sores, and the like. Typically ibuprofen compositions for oral administration in the form of pills, tablets, capsules or liquid compositions enable only about 5-10% of the ibuprofen to be actually absorbed by the body for utilization by the patient. The remaining ibuprofen binds to proteins in the body and is not available for absorption and utilization by the body. The aqueous ethanol, glycerin ibuprofen composition of the invention is easily absorbed through the oral or buccal tissue for rapid absorption.

The stable aqueous ibuprofen composition of the invention in one embodiment contains ibuprofen, glycerin, arginine and water having a pH of 7-8 and where the ibuprofen is in solution or suspension and has a particle size of 500 nm or less. The ibuprofen composition in one embodiment contains about 3-6 wt % ibuprofen, not more than 15 wt % ethanol, about 10-30 wt % glycerin and arginine in an amount to solubilize and suspend the ibuprofen in the composition. Arginine is included in an amount of about 1-10 wt % and generally in an amount of about 1-5 wt % and typically in an amount of about 1-3 wt % based on the total weight of the ibuprofen composition. In one embodiment, the composition contains about 3-4 wt % ibuprofen, not more than about 15 wt % ethanol, about 15-17 wt % glycerin, about 1-3 wt % arginine and the balance water.

Administration to the mucus membrane results in a faster uptake of the ibuprofen and active ingredients. Accordingly, any ache, pain, and/or discomfort will not reach the same severity as with gastrointestinal routes of administration due to the rapid uptake of the compositions.

### DETAILED DESCRIPTION OF THE INVENTION

The present invention provides methods and compositions that utilize small amounts of herbal medicines along with an analgesic, ibuprofen in a formulation, such as a spray formulation, characterized by dispersed particles having an average diameter of about 500 nm or less, and preferably 100 nm or less. The invention provides compositions for administration to a subject including, for example, via sublingual, buccal, or the gastrointestinal tract.

Ingredients

The present composition contains ibuprofen. Ibuprofen is a non-steroidal anti-inflammatory agent (NSAID), which is known to possess analgesic and antipyretic activities. It is useful in the treatment of pain and inflammation associated with various maladies, including the common cold, toothaches, headaches, backaches, menstrual cramps (Dysmenorrhea), the muscular aches and pains associated with Premenstrual Syndrome, rheumatoid arthritis and osteoarthritis, as well as in the reduction of fever. Ibuprofen is included in the final composition in an amount of 3-4 wt %. In another embodiment the composition contains ibuprofen in an amount of about 0.1-200 mg/ml.

The solvent/carrier for the ibuprofen composition is preferably an aqueous system containing ethanol and glycerin in amounts to solubilize the ibuprofen. Preferably the ethanol content is not more than 20 wt % and more preferably not more the 15 wt % based on the total weight of the composition. The aqueous system typically contains about 10-20 wt % ethanol, 10-30 wt % glycerin with the balance water. In one preferred embodiment, the aqueous system contains about 13-15 wt % ethanol and about 15-25 wt % glycerin and the balance water.

The ibuprofen composition contains arginine in an amount to solubilize the ibuprofen and enhance the stability of the composition. Arginine also enhances the delivery of the ibuprofen through the tissue to the patient. Arginine has been found to enhance the solubility of ibuprofen in the aqueous ethanol and glycerin system in amounts that cannot otherwise be dissolved in the aqueous ethanol glycerin system. Arginine has also been found to stabilize the ibuprofen in suspension in the aqueous ethanol glycerin system for a stable composition where the ibuprofen remains in solution and/or suspension for extended periods of time without separation or precipitation of ibuprofen or other components in the composition. Arginine further enhances absorption of ibuprofen through the membranes by dilating the blood vessels.

In one embodiment of the invention, the aqueous ibuprofen composition can optionally include feverfew in an amount to treat the patient in an amount of less than 1 wt %. Feverfew extract is derived from the feverfew plant (Tanaecetum parthenium), which is also known, for example, as Chrysanthemum parthenium, Chrisanthemum parthenium, Pyrethrum parthenium, Tanacete parthenii herba or folium, Matricaria parthenoides, Matricaria parthenium, Leucanthemum parthenium, Matricaria parthenium, Spanish pellitory, Featherfew, Featherfoil, feather-fully, and by a number of common names, various of which are used throughout the world (Midsummer daisy, Bachelor's buttons, Altamisa, nosebleed, flirtwort, ague plant, devil daisy, feddygen fenyw (Welsh), maid's weed, Missouri snakeroot, mutterkaut (German), prairie-dock, vetter-voo, wild chamomile, grande camomille (French), Santa Maria (Spain), febrifuge plant.) The extract may be obtained by techniques known in the art using solvents such as petroleum spirits or polar organic solvents. See U.S. Pat. No. 5,384,121 to Rhodes, and also WO 94 06800; EP 0 553 658; WO 92 11857; GB 2,166,952; EP 98 041; WO 98 39018. The disclosures of these patents are herein incorporated by reference.

The extract of the feverfew plant at least initially contains parthenolide, and may additionally contain other components such as Polyynes, Flavonoids and Volatile oils including camphor, bomeol and others. Feverfew also contains relatively large quantities of sesquiterpene lactones, primarily parthenolide.

In addition to parthenolide, feverfew is known to contain the following non-ubiquitous chemicals: 1-Beta-hydroxyarbusculin, 10-Epicanin, 8-Beta-reynosin, Apigenin-7-glucoside, Chrysanthemolide, Chrysanthemonin, Chrysartemin-A, Chrysartemin-B, Cosmosiin, L-Bomeol, L-camphor, Mangoliolide, Reynosin, Santamarin, Tanaparthin, Tanaparthin-1-alpha, 4-alpha-epoxide, Tanaparthin-1-beta, 4-beta-epoxide, tenetin 3-b-hydroxyparthenolide, seco-tanaparthenolide A, canin, artecanin, and balchanin.

Because feverfew extract may contain additional beneficial components, compositions comprising the extract of feverfew in the present invention as compared to compositions comprising a highly purified parthenolide that has been isolated from the additional components naturally occurring in feverfew extract.

Feverfew (Tanacetum parthenium) is an herb in the Compositae family that has been known to have therapeutic properties with mode of action based on inhibiting the release of the vasoconstrictor serotonin from platelets. Accordingly, feverfew may assist in migraine headache relief by inhibiting inflammation (e.g., via inhibiting release of inflammatory cytokines) and vasoconstriction/spasm thereby restoring normal blood flow.

Traditionally, feverfew has been administered as a raw leaf, either fresh or frozen, which is taken by chewing, by swallowing pills, tablets, capsules, by taking teas, or alcohol tinctures in which the feverfew is incorporated. It has also been administered as a tea with a concentration of 0.5-1 teaspoonfuls of feverfew per cup of boiling water. However, raw feverfew leaves are bitter and therefore unpleasant to chew and the tea is unpleasant to drink. Some evidence suggests that large amounts of feverfew cause oral ulcers or other irritations to the buccal membranes or mucosal membranes of the body including those of the mouth when taken at such high concentrations. In addition, the administration of feverfew by swallowing of the chewed material, drinking of tea, or swallowing of capsules, pills, or tinctures means that the feverfew must be released and dispersed to the central nervous system or other affected organs through the gastrointestinal system. Consequently, as discussed above, the active ingredients found in feverfew will not be readily available to a person to whom the herb has been administered. This has particularly significant drawback in the treatment of migraine headaches. In the present invention, feverfew is utilized in a powder form that has been milled or otherwise reduced in size to an average particle size of about 500 nm or less.

Ginger extract is derived from the ginger root, and may contain beneficial components in addition to gingerols, the generally recognized components of ginger extract. In the present invention, ginger can be utilized in a powder form that has been milled or otherwise reduced in size to an average particle size of about 500 nm or less. The ginger extract is included in an amount of less than 1 wt % and typically about 0.1 wt %.

In one embodiment of the present invention, the compositions of the present invention are provided in combination with a mucosal permeation enhancing agent for enhancing the mucosal absorption of the ibuprofen composition. In one example, the ibuprofen composition is a sublingual formulation. The mucosal permeation enhancer preferably can be azone, sodium glycholate, sodium cholate, sodium taurocholate, sodium taurocholate plus EDTA, deoxycholate, sodium lauryl sulfate, lauric acid, ethanol, lysophosphatidyl choline, polysorbate, cyclodextrin, cetylpyridinium chloride, cetyltrimethylammonium bromide, benzalkonium chloride, sodium salicylate, sodium EDTA, aprotinin, dextran sulfate, linoleic acid, labrafil, transcutol, urea, methoxysalicylate, POE 23 lauryl ether, various surfactants and other mucosal permeation enhancers and combinations thereof. One suitable mucosal permeation enhancer is polysorbate.

Method of Making

The present invention is also directed to a method of making the aqueous ibuprofen composition, which can be a spray formulation, with the ibuprofen and other active agents having a particle size of 500 nm or less. This particle size can be achieved by any suitable milling operation but preferably is achieved by utilizing a vacuum equipped high-shear mixer-granulator. The vacuum equipped high-shear mixer is made up of a vessel in which the mixture to be granulated is introduced that is equipped with a mixer and a mill that rotate with a normal mixer motion. The mixer and the mill have variable and adjustable speeds to ensure densification and preparation of compositions in shorter times as compared to conventional mixers. These mixers are known in the art to reduce particle size. Any like mixer may be utilized for the methods detailed herein as long as particle size reduction is achieved. The composition containing the ibuprofen and optional components such as feverfew and ginger is blended to form a fine particulate in the aqueous system. The blending process is carried out in a vacuum equipped vessel having a high shear mixer/granulator mounted thereon. Subsequently, the resulting particulates found in the formulation (spray for example) will have a size equal to or less than 500 nm (nanometers) and preferably less than about 100 nm.

The nonvolatile solvents can include, but are not limited to, the following: polyethylene glycol, propylene glycol, glycerin, vegetable oil, cotton seed oil, peanut oil, sesame oil, mineral oil, glycofurol, propylene glycol dicaprylate/dicaprate, glyceryl caprylate/caprate, oleic acid, polysorbates, sorbitan esters, caprylocaproyl macrogol-8 glycerides, ethoxydiglycol, and poloxamer block copolymers. Furthermore, cosolvency can be used to enhance the solubility of drugs in the mixed solvent system. In the preferred embodiment, the nonvolatile solvent is glycerin.

The emulsified fluid utilized in the high pressure, vacuum equipped vessel having a high shear mixer/granulator mounted thereon can be water or organic solvents such as, for instance, ethyl alcohol or other commonly used solvents, or mixtures of water and solvents.

The ibuprofen composition preferably contains an emulsifier in an amount of less than about 1 wt %, such as lecithin. The spray pharmaceutical composition also contains lecithin as the emulsifier. Egg or soy lecithin is suitable. Lecithin itself is a solid but is also available commercially as a liquid by having been mixed with oil such as soybean oil.

The aqueous ibuprofen composition can also include a sweetener to mask the taste of the ibuprofen. Sweeteners such as saccharin, aspartame (depending on the temperature used in preparation), sorbitol, corn syrup, etc. and other taste maskers such flavoring/masking agents including peppermint, orange, cherry, etc. can be included in the formulation. These sweeteners can be added to any one or more of the components of the composition to insure their effectiveness throughout the composition.

The aqueous ibuprofen composition of the invention preferably contains a buffering agent. Arginine is added as a buffering agent to modify the pH of the composition. The ideal pH of the final composition will be approximately 7.0-8.0. Additionally, arginine enhances the solubility and stability of ibuprofen within the final composition.

The resulting ibuprofen produced by the process of the invention is soluble in an aqueous medium form the stable aqueous ibuprofen composition of the present invention where the ibuprofen remains in solution or suspension for extended periods of time. In one embodiment, the ibuprofen composition is stable for at least about 6 months. The process for forming the water-soluble ibuprofen begins by dissolving ibuprofen USP grade powder in a dissolution solution in succession or sequential manner such as a process according to the Homeopathic Pharmacopeia of the United States (HPUS), thereby forming a homeopathic premix. HPUS is a well-known manual that details homeopathic processing. One skilled in the art would know to use the manual as reference for producing homeopathic remedies, and is incorporated by reference.

The ibuprofen is dissolved using any suitable solvent. The ibuprofen is initially dissolved in a pharmaceutically acceptable solvent. A particularly suitable solvent for forming the ibuprofen solution is ethanol. In one embodiment, the dissolution solution contains a mixture of water, alcohol, and glycerin. An example of the ibuprofen homeopathic premix is listed in Table 1. After forming the ibuprofen homeopathic premix, the premix is transferred to a vacuum equipped vessel having a high shear mixer mounted thereon. The ibuprofen homeopathic premix is mixed in the mixer for approximately 10-30 minutes. In one embodiment the mixture is mixed for approximately 15 minutes.

After mixing, the ibuprofen homeopathic premix is rested for approximately 10-20 minutes, preferably for approximately 15 minutes. This is followed by equilibrating the mixed ibuprofen homeopathic premix for approximately 24 hours. After equilibrating, the mixed ibuprofen homeopathic premix may be filtered through a filtration assembly, such as a sieve (10 micron sieve preferred). Through the mixing process, the particle size of the ibuprofen homeopathic premix will be reduced a size equal to or less than 500 nanometers (nm) and preferably less than 100 nm.

The present invention is also directed to a process for preparing a sublingual spray pharmaceutical composition where the particle size of the composition is less than or equal to 500 nm. The process for forming the water-soluble ibuprofen begins by dissolving ibuprofen USP grade powder in a dissolution solution in succession manner according to the Homeopathic Pharmacopeia of the United States (HPUS), thereby forming a homeopathic premix. As detailed above, HPUS is a well-known manual that details homeopathic processing. The ibuprofen is dissolved using any suitable solvents. In one embodiment, the dissolution solution contains water, alcohol, glycerin or a combination thereof.

The homeopathic premix is then transferred to a first container. After transferring, at least one homeopathic ingredient is added to the first container. The homeopathic ingredients include feverfew premix, ginger premix or a combination thereof. Preferably, a combination of feverfew and ginger is added to the homeopathic premix. Tables 2 shows the ingredients of the ginger premix, which involves dissolving ginger powder in a solvent solution including ethyl alcohol and water. This process is well-known and detailed in HPUS. Table 3 shows the ingredients of the feverfew premix, which involves dissolving feverfew powder in a solvent solution including ethyl alcohol and water. This process is well-known and detailed in HPUS.

This is followed by, in a second container, dissolving salt, sugar and at least one preservative in a solution of water, glycerin or a combination thereof. Lecithin (an emulsifier as detailed above), polysorbate or a combination thereof is added to the second container. This mixture is mixed in the vacuum equipped vessel having a high shear mixer mounted thereon under 5-20 psi and 2500-4000 RPM for approximately 5-15 minutes. Preferably, the high shear mixing will be done under 10-14 psi and 3500 RPM for approximately 10 minutes.

The mixed contents of the second container are then added to the first container containing the homeopathic premix with homeopathic ingredients. The mixture is the mixed in the high shear mixer for approximately 5-15 minutes and preferably for 10 minutes. After mixing, flavoring agents, as detailed above are added to the mixture along with arginine. After adding the flavoring agents and arginine, the mixture is mixed under the high shear mixer for approximately 5-15 minutes. After mixing, the mixture is rested for approximately 10-20 minutes, preferably for approximately 15 minutes. This is followed by equilibrating the mixture for approximately 24 hours. After equilibrating, the mixture is filtered through a filtration assembly, such as a sieve (10 micron sieve preferred). Through the mixing process, the particle size of the sublingual spray pharmaceutical composition will be reduced a size equal to or less than 500 nanometers (nm). The overall sublingual spray pharmaceutical composition is detailed in Table 4 based on a 100% w/w solution.

TABLE 1

| INGREDIENT | UNIT OF MEASURE | AMOUNT PER 18 LITERS |
|---|---|---|
| Ibuprofen | kg (kilograms) | 2 |
| powder | | |
| Ethyl Alcohol | l (liters) | 10 |
| Glycerin | l (liters) | 7.600 |
| Water | l (liters) | 0.400 |

TABLE 2

| INGREDIENT | UNIT OF MEASURE | AMOUNT PER 4 LITERS |
|---|---|---|
| Ginger powder | g (grams) | 400 |
| Ethyl Alcohol | l (liters) | 2.6 |
| Water | l (liters) | 1.4 |

TABLE 3

| INGREDIENT | UNIT OF MEASURE | AMOUNT PER 4 LITERS |
|---|---|---|
| Feverfew | g (grams) | 400 |
| powder | | |
| Ethyl Alcohol | l (liters) | 2.6 |
| Water | l (liters) | 1.4 |

TABLE 4

| INGREDIENTS | % w/w |
|---|---|
| Water | 46.2500 |
| Glycerin (99.5% | 4.0000 |
| USP) | |
| Salt (NaCl-39.34% | 0.3000 |
| Na) | |
| Sucralose | 0.3500 |
| Lecithin | 3.0000 |
| Polysorbate | 0.6000 |
| Potassium Sorbate | 0.1500 |
| (granular) | |
| Ibuprofen (premix | 35.0000 |
| from Table 1) | |
| Ginger (premix | 0.3500 |
| from Table 2) | |
| Feverfew (premix | 3.5000 |
| from Table 3) | |
| Masking flavor | 2.0000 |
| Cherry flavor | 2.5000 |
| Arginine | 2.0000 |
| TOTAL | 100.0000 |

In another embodiment of the invention, the aqueous ibuprofen composition is prepared by forming a first mixture of water and glycerin as the aqueous medium for the final composition. An emulsifier and arginine are then added to the first mixture and mixed a medium speed in a homogenizer mixing device for about 15 minutes until dissolved. In a separate mixing device, an ibuprofen solution is prepared by mixing ibuprofen in ethanol to dissolve the ibuprofen. The resulting ibuprofen solution then added to the first mixture in the mixing device and continuously mixed under a vacuum for about 15 minutes to obtain the stable aqueous ibuprofen composition. In one embodiment resulting aqueous composition contains about 3-4 wt % ibuprofen and the balance being water, glycerin, ethanol, arginine and other additives. In this embodiment, the final aqueous ibuprofen composition contains about 15 wt % glycerin, about 15 wt % ethanol, about 2 wt % arginine, about 3.48 wt % ibuprofen and the balance water.

In another embodiment of the invention, sublingual spray composition is prepared in the form of a stable aqueous ibuprofen composition that can be administered directly the mucosa. The composition contains a suitable buffering agent, mucosal permeation enhancers and stabilizers. The following examples are intended to be exemplary of the invention.

In one embodiment of the invention, an aqueous ethanol ibuprofen composition is a clear solution that can be administered through the mucal and/or buccal tissue for delivering ibuprofen through the tissue to the bloodstream. An ibuprofen solution is prepared by mixing ibuprofen, ethanol and water followed by mixing to form a clear solution. The mixture can be mixed by a high shear mixer at about 3500 rpm under high shear and under vacuum to obtain the clear solution. The resulting clear solution can contain about 10 wt % ibuprofen, 60 wt % ethanol and 30 wt % water. A mixture of water, glycerin and arginine are added to the clear ibuprofen solution and mixed under high shear in a high shear mixer under vacuum to obtain the stable ibuprofen composition. The resulting composition can be filtered to obtain a clear solution that is stable for about 6 months without separation or precipitation. The arginine is included in an amount to enhance the solubility of the ibuprofen and stabilize the resulting mixture.

The stable aqueous ethanol ibuprofen composition contains arginine in an amount to maintain the ibuprofen in solution or suspension. The ibuprofen composition contains about 1 to 20 wt %, preferably 0.5 to 10 wt %, and most preferably about 2 to 6 wt % ibuprofen in solution or suspension. The high shear mixing under vacuum produces the clear ibuprofen composition where the ibuprofen is reduced to a particle size of 500 nm or less, and preferably 100 nm or less. Arginine is preferably included in an amount of about 2 wt % but can be included in an amount of 1 to 10 wt % based on the total weight of the ibuprofen composition. The ibuprofen composition preferably contains not more than 15 wt % alcohol. In one embodiment, the ibuprofen composition can contain about 13 to 17 wt %, and preferably about 13 to 15 wt % ethanol. The ibuprofen can contain glycerin as a cosolvent in an amount of about 16 wt % based on the total weight of the composition, although glycerin can be included in an amount of about 10 to 30 wt % based on the total weight of the ibuprofen composition. The resulting composition is believed to contain ibuprofen is solution and as a stable micro suspension. The arginine is believed to enhance the solubility of ibuprofen in an aqueous system and to stabilize the micro suspension of ibuprofen composition without separation or precipitation of ibuprofen for up to about 6 months where the ibuprofen composition contains about 3-6 wt % ibuprofen and not more than 20 wt % ethanol and preferably not more than 15 wt % ethanol.

### Example 1

A mixture of deionized water and glycerin are formed in a mixer until a uniform mixture is obtained. Salt, sucralose, lecithin, a polysorbate/POLYSORBATE 20 blend and potassium sorbate are then added to the mixture and continuously mixed to form a uniform first mixture. In a separate mixer ibuprofen is dissolved in ethanol (95 wt %) by mixing to for form a clear alcohol solution. The ibuprofen alcohol solution is then added to a high shear mixer with the first mixture. The resulting mixture is mixed in the high shear mixer a 3500 rpm under a vacuum for about 15 minutes. The speed of the mixer is then reduced and mixing in continued for about 30 minutes. A flavoring agent, arginine and a cherry flavor are then added to the mixer and mixing is continued for about 30 minutes. The final aqueous ibuprofen composition had the formula as shown in Table 5.

TABLE 5

| | |
|---|---|
| Water (deionized) | 54.22 wt % |
| Glycerin (99.5% USP) | 16.00 wt % |
| Salt (sodium chloride) | 0.30 wt % |
| Sucralose | 0.35 wt % |
| Lecithin | 3.00 wt % |
| Polysorbate/POLYSORBATE 20 | 0.15 wt % |
| Ethanol (95% vol) | 15.00 wt % |
| Arginine | 2.00 wt % |
| Ibuprofen | 3.48 wt % |

Balance water, flavorings and other additives

### Example 2

In this example, an aqueous ibuprofen composition is obtained that can be used as a sublingual spray composition. A mixture of deionized water, glycerin, feverfew powder, ginko bilboa powder and ginger root powder were combined and mixed in a triple motion mixing vessel and mixed to obtain a uniform first mixture. When the uniform mixture is obtained salt, sucralose lecithin, polysorbate, POLYSORBATE 20, and potassium sorbate are added to the first mixture and continuously mixed until the components are uniformly mixed to obtain a second mixture. In a separate mixer, ibuprofen is dissolved in ethanol to form a clear third mixture. The third mixture of ibuprofen is then added to the second mixture and mixed in a high shear mixer at 3500 rpm and under vacuum for about 15 minutes to form a uniform mixture. The speed of the mixer is reduced and mixing is continued for about 30 minutes. A masking flavor, arginine and a cherry flavor are then added and continuous mixed for under vacuum for 30 minutes. The resulting mixture is the filtered to obtain the stable aqueous ibuprofen composition. The resulting ibuprofen composition is stable for about 6 months with no separation, settling or precipitation of the components. The final composition had the formula shown in Table 6.

TABLE 6

| | |
|---|---|
| Water (deionized) | 54.22 wt % |
| Glycerin (99.5% USP) | 16.00 wt % |
| Feverfew powder | 0.20 wt % |
| Gingko Bilboa powder (24% Ginkosides) | 0.10 wt % |
| Ginger Root (ground powder) | 0.10 wt % |
| Salt (sodium chloride) | 0.30 wt % |
| Sucralose | 0.35 wt % |
| Lecithin | 3.00 wt % |
| Polysorbate/POLYSORBATE 20 | 0.15 wt % |
| Potassium sorbate | 0.15 wt % |
| Ethanol (95% vol) | 15.00 wt % |
| Ibuprofen | 3.48 wt % |
| Masking flavor | 2.00 wt % |
| Arginine | 2.00 wt % |
| Cherry flavor | 2.5 wt % |

### Example 3

An ibuprofen mixture containing 10 wt % ibuprofen, 60 wt % ethanol, and 30 wt % water is prepared by combining the ingredients and mixing in a high shear mixer under a vacuum until a clear solution is obtained. Additional water, glycerin and arginine are added to the ibuprofen mixture and mixed under high shear and under a vacuum. The resulting composition containing about 2.0 wt % arginine is a clear aqueous ethanol solution that is stable for about 6 months. The final composition contained about 3 wt % ibuprofen, 2 wt % arginine, about 16 wt % glycerin, about 15 wt % ethanol and the balance water.

Method of Use

The invention provides methods and composition for administration, via sublingual, buccal, or the gastrointestinal tract. Sublingual administration offers advantages over other routes of administration. For example, compositions administered to the sublingual space have a rapid onset of action, reach high levels in the blood, avoid the first-pass effect of hepatic metabolism, and avoid exposure of the drug to fluids of the gastrointestinal tract. Additional advantages include easy access to the mucus membrane of the sublingual space so that an active substance contained in a therapeutic composition can be easily applied and localized. Further, there is good potential for prolonged delivery through the sublingual mucosal membrane.

The sublingual mucosa includes the membrane of the ventral surface of the tongue and the floor of the mouth. The sublingual mucosa is permeable, thus giving rapid absorption and acceptable bioavailability of many active substances. Furthermore, the sublingual mucosa is convenient, accessible, and generally well accepted. This route has been investigated clinically for the delivery of a substantial number of drugs.

Accordingly, in one aspect of the invention, the present spray formulation is designed for delivery to the sublingual mucosa, however, as detailed above, the composition may be administered via the buccal route and gastrointestinal tract. Spray administration containers for various types of sublingual sprays are known and typically will be suitable for the invention. The composition will commonly be contained in a small bottle or similar container with a focused nozzle from which the composition can be dispersed as a fine mist to be directed under the tongue. Using ambient air as the propelling agent, one can have the bottle made of a flexible plastic, so that merely squeezing the bottle's side propels the spray out through the nozzle into the sublingual space. Air is also the propelling agent for a pump sprayer, in which the user manipulates a small pump button which pumps air into the container and causes the liquid spray to be emitted on the return stroke. Alternatively, the bottle can be pressurized with a gas that is inert to the user and to the ingredients present in the composition. The gas will be dissolved under pressure in the container or may be generated by dissolution or reaction of a solid material that forms the gas as a product of dissolution or as a reaction product. Typical gases, which can be used, include nitrogen, argon, and a carbon dioxide.

Typically a subject will spray three to ten sprays at each administration of the sublingual spray pharmaceutical composition, with the administration being repeated on an as needed basis. During use a subject need merely raise their tongue and direct a spray comprising the formulation of the invention to the space under the tongue. The frequency of administration will be dependent on the nature of the usage. If administration is for relief of a current condition, such as a current headache, migraine, toothache, earache, and the like, initial relief effects can be expected within a few minutes of administration. Dosages may be repeated at intervals as the effect wears off, if the headache, migraine, toothache, or earache persists. The user will normally discontinue administration once the headache, toothache, or earache subsides. Administration can be resumed at a subsequent time when another headache, migraine, toothache, earache, or the like occurs.

In another aspect, the compositions of the invention may be administered at similar or smaller dosages and on a regular or less frequent basis to treat body aches or arthritic pain. The sublingual spray delivering system can be a unit dose delivery system. The volume of solution or suspension delivered per dose can be from 5 to 600 μl (microliters), generally about 50-400 μl, and typically between 50 to 250 μl. Delivery quantities of the spray pharmaceutical composition for sublingual use are typically about 200 μl per spray.

While the invention has been described in connection with what is presently considered to be the most practical and preferred embodiment, it is to be understood that the invention is not to be limited to the disclosed embodiments, but on the contrary, is intended to cover various modifications and equivalent arrangements included within the spirit and scope of the appended claims.

What is claimed is:

1. A stable water-soluble ibuprofen pharmaceutical composition which is stable for extended periods and through a wide variety of temperature extremes, effective for use as a sublingual spray comprising:
a solution of 0.1-200 mg/ml of ibuprofen USP grade powder, dissolved in an aqueous system of water, alcohol and glycerin;
wherein said composition is stable without separation, precipitation or settling for at least 6 months; and
wherein the particle size of said ibuprofen in said stable sublingual spray pharmaceutical composition is less than or equal to 500 nanometers.

2. The composition of claim 1, wherein the particle size of said ibuprofen in said stable sublingual spray pharmaceutical composition is less than or equal to 100 nanometers.

3. The composition of claim 1, wherein said aqueous system contains about 10-20 wt % alcohol, and about 10-30 wt % glycerin with the balance water.

4. The composition of claim 3, wherein said aqueous system contains about 13-15 wt % alcohol, and about 15-25 wt % glycerin with the balance water.

5. The composition of claim 1, wherein said alcohol is ethyl alcohol.

6. The composition of claim 3, wherein said alcohol is ethyl alcohol.

7. The composition of claim 4, wherein said alcohol is ethyl alcohol.

8. The composition of claim 1, further including about 0.3% wt/wt sodium chloride.

9. The composition of claim 1, further including a sweetener.

10. The composition of claim 9, wherein said sweetener is sucralose in an amount of about 0.35% wt/wt.

11. The composition of claim 1, further including arginine in an amount effective to result in a pH of approximately 7.0-8.0.

12. The composition of claim 1, further including a flavoring agent.

13. The composition of claim 1, further including a preservative.

14. The composition of claim 1, further comprising feverfew and ginger extract.

15. The composition of claim 1, further comprising an emulsifier.
